(19) United States
(12) Patent Application Publication
(10) Pub. No.: US 2008/0153728 A1
(43) Pub. Date: Jun. 26, 2008

(54) Cleansing compositions having improved fragrance characteristics and methods for the formulation thereof

(75) Inventors: Robert Dail, Phoenix, AZ (US); Juan Salas, Mesa, AZ (US)

Correspondence Address: THE DIAL CORPORATION, 15501 N. DIAL BOULEVARD, SCOTTSDALE, AZ, 85260, US

(73) Assignee: The Dial Corporation

(21) Appl. No.: 12/012,555

(22) Filed: Feb. 4, 2008

Related U.S. Application Data
(63) Continuation-in-part of application No. 11/465,581, filed on Aug. 18, 2006.
(60) Provisional application No. 60/709,718, filed on Aug. 19, 2005.

(51) Int. Cl. A61K 8/18

(52) U.S. Cl. 510/130

(57) ABSTRACT

Methods for formulating a cleansing composition having a desired fragrance profile are provided. In one embodiment, the method includes the step of determining a quantity of filler to be included in the cleansing composition wherein the quantity of filler is about 1% to about 10% by weight of the cleaning composition (WCC). The method also includes the step of selecting a first fragrance quantity to be included in the cleansing composition. The first fragrance quantity is selected to be about 1% to about 10% less than a second fragrance quantity, expressed as a percentage by WCC, necessary to substantially achieve the desired fragrance profile if the cleansing composition comprised less than about 1% by WCC of filler.

### CROSS-REFERENCES TO RELATED APPLICATIONS

The present application is a continuation-in-part of U.S. application Ser. No. 11/465,581, filed Aug. 18, 2006, which claims the benefit of U.S. Provisional Application No. 60/709718, filed Aug. 19, 2005, which is herein incorporated in its entirety by reference.

### TECHNICAL FIELD

The present invention relates generally to cleansing compositions and, more particularly, to cleansing compositions having improved fragrance characteristics.

### BACKGROUND

Various types of cleansing compositions have been developed for topical application to human skin (e.g., toilet bar soaps, liquid body soaps, etc.), for cleaning articles of clothing and other textiles (e.g., laundry detergent bars, powered laundry detergent, etc.), and for use on a variety of household surfaces. Manufacturers commonly add perfume or fragrance to cleansing compositions of all types to provide their products with desired fragrance profiles. The particular fragrance profile of a cleansing product is of significant commercial importance. A carefully-engineered fragrance may connote to the user a number of desirable qualities, such as cleanliness and freshness. In addition, a unique fragrance profile may distinguish one cleaning composition from its competitors.

Manufacturers have long recognized the importance of providing cleansing compositions, especially personal care cleansing compositions, with desirable fragrance profiles. Considerable expense is incurred in developing unique and desirable fragrances, which typically comprise complex mixtures of synthetic and/or natural oils. Furthermore, fragrance is relatively costly to produce. The quantity of fragrance utilized to a produce a given cleansing product, such as a toilet bar soap, thus becomes a significant driver in determining the overall cost of production.

Accordingly, it is desirable to provide cleansing compositions, especially personal care cleansing compositions, having enhanced fragrance characteristics. In particular, it is desirable to provide cleansing compositions that achieve desirable fragrance profiles utilizing minimal quantities of fragrance. It is also desirable to provide a method for formulating such cleansing compositions. Other desirable features and characteristics of the present invention will become apparent from the subsequent detailed description of the invention and the appended claims, taken in conjunction the accompanying drawing and this background of the invention.

### BRIEF DESCRIPTION OF THE DRAWINGS

At least one example of the present invention will hereinafter be described in conjunction with the following figure, wherein like numerals denote like elements, and:

FIG. 1 is flowchart illustrating a method for formulating a cleansing composition having a desired fragrance profile in accordance with an exemplary embodiment of the present invention.

### BRIEF SUMMARY

Methods for formulating a cleansing composition having a desired fragrance profile are provided. In one embodiment, the method includes the step of determining a quantity of filler to be included in the cleansing composition wherein the quantity of filler is about 1% to about 10% by weight of the cleaning composition (WCC). The method also includes the step of selecting a first fragrance quantity to be included in the cleansing composition. The first fragrance quantity is selected to be about 1% to about 10% less than a second fragrance quantity, expressed as a percentage by WCC, necessary to substantially achieve the desired fragrance profile if the cleansing composition comprised less than about 1% by WCC of filler.

Cleansing compositions with enhanced fragrance characteristics are also provided. In accordance with an exemplary embodiment, a cleansing composition having a fragrance profile includes about 1% to about 10% by weight of the cleansing composition (WCC) of filler, and a first quantity of fragrance that is less than a second quantity of fragrance necessary to substantially achieve the fragrance profile of if the cleansing composition comprised less than about 1% by WCC of filler.

In accordance with a further exemplary embodiment, a personal care cleansing composition having enhanced fragrance characteristics is provided. The personal care cleansing composition includes about 68% to about 83% by weight of the personal care cleansing composition (WCC) of surfactant, about 10% to about 15% by WCC of water, about 0.5% to about 2.0% by WCC of fragrance, and about 1.0% to about 8.0% by WCC of talc.

### DETAILED DESCRIPTION

The following detailed description of the invention is merely exemplary in nature and is not intended to limit the invention or the application and uses of the invention. Furthermore, there is no intention to be bound by any theory presented in the preceding Background or the following Detailed Description.

The present inventors have discovered that the fragrance characteristics of fragrance profiles of cleansing compositions may be significantly enhanced through the addition of certain quantities of filler, such as talc. As appearing herein, the term “fragrance profile” is utilized to describe aspects of the composition's overall bouquet as perceived by the end user and indicated by, for example, testing data. Such aspects of the composition's overall bouquet may include, but are not limited to, fragrance intensity and longevity. Surprisingly, the enhanced fragrance characteristics of the cleansing compositions of the present invention are exhibited over a relatively limited range of fillers. According to testing data, statistically-significant improvements in fragrance characteristics first appear at quantities of about 1% by weight of the cleansing composition (WCC) of filler and significantly diminish, or entirely disappear, at quantities exceeding about 10% by WCC of filler. As a result of the enhanced fragrance characteristics and the presence of filler, the quantity of fragrance in the cleansing composition can be decreased from an original fragrance quantity, expressed as percentage by WCC, that is necessary to obtain the same fragrance profile if less than 1% by WCC of filler is present. In an exemplary embodiment, the fragrance can be decreased by about 1% to about 10% relative to the original fragrance quantity without negatively impacting the composition's overall fragrance profile. For example, testing data shows that users are generally unable to detect a change in fragrance profile of a preferred cleansing composition containing about a 5% decrease in fragrance quantities when combined with about a 5% increase by WCC of filler.

In a preferred embodiment, the percentage decrease in the quantity of fragrance and the percentage increase by WCC of filler conveniently differ by no more than about 3% and are preferably substantially equivalent. That is, taking the cleansing composition to contain a predetermined quantity of fragrance that is X% less than an original fragrance quantity (expressed as percentage by WCC), the quantity of filler contained within the cleansing composition (expressed as a percentage by WCC) differs from X% by no more than about 3; and, in a more preferred embodiment, the quantity of filler contained within cleansing composition is substantially equivalent to X%.

Embodiments of the cleansing compositions described herein are by no means limited to personal care cleansing compositions and may include, for example, compositions useful for cleaning textiles, such as laundry detergent bars, powered laundry detergents, and the like. This notwithstanding, in a preferred group of embodiments, the cleansing composition assumes the form of a personal care cleansing composition, such as a toilet bar soap. As will be appreciated by one skilled in the art, such personal care cleansing compositions generally contain one or more aromatic surfactants, such as soaps (i.e., monovalent salts of monocarboxylic fatty acids), synthetic detergents, or a combination of synthetic detergents and soaps (as in the case of combars). If the personal care composition contains one or more soaps, the soaps may each include: (i) a long fatty acid chain having alkyl chain links ranging from about 8 carbons to about 18 carbons in length, and (ii) one or more types of counter-ions, such as sodium ions, potassium ions, ammonium, alkanol ammonium, and the like. The personal care composition may be further provided with a number of additives that impart desirable characteristics to the composition not inherent in the soap itself. Moisturizers, for example, may be added to treat skin dryness; humectants may be added to promote moisture retention; antibacterial agents may be added for disinfection purposes; dyes may be added for color; etc.

As previously stated, enhanced fragrance characteristics are observed at quantities ranging from about 1% to about 10% by WCC of filler. However, in the exemplary case wherein the cleansing composition assumes the form of a personal care cleansing composition (e.g., a toilet bar soap), it is preferable that the personal care cleansing composition contains filler quantities of about 1% to about 6% by WCC of filler. In a more preferred embodiment, the cleansing composition contains filler quantities of about 3% to 5% by WCC of filler. In a still more preferred embodiment, the cleansing composition contains filler quantities of about 4% to about 5% by WCC of filler. Embodiments of personal care cleansing compositions exceeding approximately 6% by WCC of filler have been found to exhibit negative skin feel characteristics (i.e., various attributes appreciable to a user during and after application of the personal care cleansing composition, typically in a wet state, to the skin). Similarly, in embodiments wherein the cleansing composition assumes the form of a personal care cleansing product, it is preferred that the composition contains about 0% by WCC of grease solvents to avoid negatively impacting the composition's skin feel characteristics.

Filler Components

A wide variety of inert materials, both organic and inorganic, are suitable for use as filler in the above-described cleansing compositions. It is preferred that the selected filler is generally chemically inert and does not have an odor that is humanly perceptible. If the cleansing composition assumes the form of a laundry bar, powered laundry detergent, or other product that is not applied to human skin, the cleansing composition may employ talc, clays, fumed silica, calcium carbonate, starch, sugar, and various combinations of the foregoing materials as fillers. However, if the cleansing composition assumes the form of a personal care cleansing product (e.g., a toilet bar soap), it is generally preferred that overly-abrasive materials (e.g., calcium carbonate) are not utilized as fillers. Examples of suitable fillers for use in personal care cleansing composition include talc, fumed silica, and starch. In a preferred embodiment, the filler comprises talc and, ideally, consists substantially entirely of talc. As appearing herein, the term “talc” denotes a composition consisting entirely or almost entirely of hydrated magnesium silicate. Talc may generally be described by either of the following formulas: H₂Mg₃(SiO₃)₄ or Mg₃Si₄O₁₀(OH)₂. Preferably, talc in the form of talcum powder is utilized. Talc is the preferred filler due, at least in part, to its lubricating effects and to its ability to increase the overall moisture content of the personal care cleansing composition. The addition of talc results in enhancement of a number of skin feel characteristics (e.g., the production of noticeably creamier lather). Without intending to be bound by theory, this is believed to be the result of an increase in the surface area of the personal care composition, in bar form, when granules of talc are separated from the bar thus producing tiny cavities in the bar's outer surface into which water may flow.

Exemplary Method of Formulating a Cleansing Composition

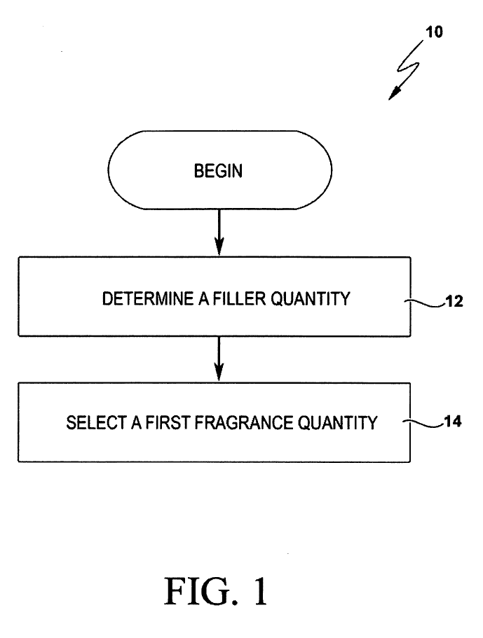

Embodiments of a method for formulating an exemplary cleansing composition having a desired fragrance profile are also provided herein. For example, and with reference to FIG. 1, an exemplary method 10 includes the step of determining a quantity of filler to be included in the cleansing composition (step 12). In a preferred embodiment, the quantity of filler is determined to be about 1% to about 10% by WCC of filler. In a more preferred embodiment, the quantity of filler is determined to be about 1% to about 6% by WCC of talc. In a still more preferred embodiment, the quantity of filler is determined to be about 5% by WCC of talc.

Referring still to FIG. 1, method 10 further includes the step of selecting a first fragrance quantity to be included in the cleansing composition (step 14). In a preferred embodiment, the first fragrance quantity is about 1% to about 10% less than a second fragrance quantity, expressed as a percentage by WCC, that is necessary to substantially achieve the desired fragrance profile if the cleansing composition comprised less than about 1% by WCC of filler. In a more preferred embodiment, the first fragrance quantity is chosen to be approximately 5% less than the second fragrance quantity expressed as a percentage by WCC.

Exemplary Embodiments of a Personal Care Cleansing Composition

By way of illustration and not of limitation, the formulae of two exemplary personal care cleansing compositions are set-forth in the following table, along with a formulation for a comparative, conventional personal care cleansing composition.

| | Comparative | Example 1 | Example 2 |
|---|---|---|---|
| Component | Weight % | Weight % | Weight % |
| Sodium Soap | 79.790 | 74.760 | 72.140 |
| Water | 11.670 | 11.660 | 11.690 |
| Fragrance (Parfum) | 1.500 | 1.425 | 1.380 |
| Talc | 0.000 | 5.080 | 8.000 |
| Other (glycerin, colorants, | 7.040 | 7.070 | 6.790 |
| PEG, etc.) | | | |

The foregoing table presents the formula for two exemplary embodiments of a personal care cleansing product, such as a toilet bar soap. The foregoing table also includes the formula for a comparative, conventional toilet bar soap having a fragrance profile substantially identical to that of the exemplary bar soap, as perceivable by an end user. As can be seen, the comparative toilet bar soap contains nearly 80% by WCC of sodium soap, over 11% by WCC of water, and 1.5% by WCC of fragrance. Notably, the comparative toilet bar soap does not contain filler. By comparison, the first exemplary bar soap contains nearly 75% by WCC of sodium soap and approximately 5% by WCC of filler, which, in this exemplary case, consists entirely of talc. The first exemplary bar soap also contains 1.425% by WCC of fragrance. The first exemplary bar soap is able to achieve a fragrance profile that is substantially identical to that of the comparative bar soap utilizing a fragrance quantity that is less than the fragrance quantity contained within the comparative bar soap. Specifically, the quantity of fragrance contained within the first exemplary bar soap (i.e., 1.425% by WCC of fragrance) is 5% less than the quantity of fragrance contained within the comparative bar soap (i.e., 1.5% by WCC of fragrance). Similarly, the second exemplary bar soap, which is also able to achieve a fragrance profile substantially identical to that of the comparative bar soap, contains about 72% by WCC of sodium soap, 8% by WCC of filler (e.g., talc), and about 8% less fragrance than does the comparative bar soap. It should be noted that, in the foregoing exemplary formulas, the sodium soap may or may not be super-fatted. If super-fatted, the sodium soap may contain, for example, approximately 5% by WCC of fatty acids, such as coconut acid, palm acid, tallow acid, and the like.

Without intending to be bound by theory, the present inventors believe that the presence of a substantially odorless filler, such as talc, to the cleansing composition enhances the composition's fragrance profile by increasing the quantity of fragrance contained within composition relative to the composition's other aromatic contents, particularly aromatic surfactants (e.g., soaps and synthetic detergents). With respect to the foregoing example, the presence of talc within the first exemplary bar soap results in a lesser percentage by WCC of other components relative to the comparative bar soap. For example, the first exemplary bar soap employs about 5% less sodium soap (expressed as a percentage by WCC) than does the comparative bar soap. Consequently, the ratio of fragrance-to-soap (and other such aromatic components) within the first exemplary bar soap is increased relative to the comparative bar soap. Similarly, the first exemplary bar soap contains a larger quantity of fragrance per a given volume of soap than does the comparative bar soap. Therefore, the first exemplary bar soap achieves substantially same the fragrance profile utilizing a minimal quantity of fragrance.

The quantity of fragrance contained within embodiments of the cleansing composition may also be described in terms of a fragrance-to-surfactant ratio. For example, in the above-described example wherein a cleansing composition contains about 1% to about 10% by WCC of filler and a first quantity of fragrance that is less than a second quantity of fragrance necessary to substantially achieve the fragrance profile if the cleansing composition comprised less than about 1% by WCC of filler, the cleansing composition may further have a first fragrance-to-surfactant ratio that is substantially equivalent to a second fragrance-to-surfactant ratio exhibited by the cleansing composition if the cleansing composition were to lack filler and contain the second quantity of fragrance. Similarly, an exemplary method for formulating a cleansing composition may include the steps of: (1) determining a quantity of filler to be included in the cleansing composition wherein the quantity of filler is about 1% to about 10% by WCC; and (2) choosing a fragrance quantity to be included in the cleansing composition. The fragrance quantity is chosen such that the ratio of fragrance-to-surfactant is maintained with respect to a formulation of the cleansing composition lacking the determined quantity of filler.

There has thus been provided multiple exemplary cleansing compositions, including several personal care cleansing compositions, having enhanced fragrance characteristics. Embodiments of the cleansing compositions described above achieve desirable fragrance profiles utilizing minimal quantities of fragrance. There has also been provided examples of a method for formulating embodiments of such cleansing compositions.

Embodiments of the cleansing compositions described herein are by no means limited to personal care cleansing compositions and may include, for example, compositions useful for cleaning textiles, such as laundry detergent bars, powered laundry detergents, and the like. This notwithstanding, in a preferred group of embodiments, the cleansing composition assumes the form of a personal care cleansing composition, such as a toilet bar soap. As will be appreciated by one skilled in the art, such personal care cleansing compositions generally contain one or more soaps (i.e., monovalent salts of monocarboxylic fatty acids), synthetic detergents, or a combination of synthetic detergents and soaps (as in the case of combars). Furthermore, such personal care cleansing compositions are preferably substantially free of agents (e.g., grease solvents) that may cause skin irritation. In a generalized group of embodiments, a personal care cleansing composition contains about 68% to about 83% by WCC of surfactant (e.g., sodium soap), about 10% to about 15% by WCC of water, about 0.5% to about 2.0% by WCC of fragrance, about 1.0% to about 8.0% by WCC of talc, and about 6.0% to about 7.5% by WCC of additional ingredients (e.g., glycerin, colorants, PEG, etc.). In a more preferred group of embodiments, a personal care cleansing composition contains about 70% to about 80% by WCC of surfactant (e.g., sodium soap), about 11% to about 13% by WCC of water, about 1.0% to about 1.5% by WCC of fragrance, about 4.0% to about 6.0% by WCC of talc, and about 6.0% to about 7.0% by WCC of additional ingredients (e.g., glycerin, colorants, PEG, etc.)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for formulating a cleansing composition having a desired fragrance profile, the method comprising:
determining a quantity of filler to be included in the cleansing composition, the quantity of filler being about 1% to about 10% by weight of the cleaning composition (WCC); and
selecting a first fragrance quantity to be included in the cleansing composition, the first fragrance quantity being about 1% to about 10% less than a second fragrance quantity, expressed as a percentage by WCC, necessary to substantially achieve the desired fragrance profile if the cleansing composition comprised less than about 1% by WCC of filler.

2. A method according to claim 1, wherein the step of determining comprises establishing a quantity of talc to be included in the cleansing composition, the quantity of talc being about 1% to about 6% by WCC.

3. A method according to claim 1, wherein the step of determining comprises establishing a quantity of about 5% by WCC of talc to be included to the cleansing composition.

4. A method according to claim 3, wherein the step of selecting comprises choosing a first fragrance quantity to be included in the cleansing composition that is approximately 5% less than the second fragrance quantity expressed as a percentage by WCC.

5. A method according to claim 1, wherein the step of selecting comprises choosing a first fragrance quantity to be included in the cleansing composition, the first fragrance quantity chosen such that a fragrance-to-surfactant ratio of the cleansing composition is maintained with respect to a formulation of the cleansing composition lacking the determined quantity of filler.

6. A cleansing composition having a fragrance profile, the cleansing composition comprising:
about 1% to about 10% by weight of the cleansing composition (WCC) of filler; and
a first quantity of fragrance that is less than a second quantity of fragrance necessary to substantially achieve the fragrance profile if the cleansing composition comprised less than about 1% by WCC of filler.

7. A cleansing composition according to claim 5, wherein the filler comprises talc.

8. A cleansing composition according to claim 7, wherein the filler consists substantially entirely of talc.

9. A cleansing composition according to claim 6, wherein the first quantity of fragrance is about 1% to about 10% less than the second quantity of fragrance expressed as a percentage by WCC.

10. A cleansing composition according to claim 9, wherein the first quantity of fragrance is about 5% less than the second quantity of fragrance expressed as a percentage by WCC.

11. A cleansing composition according to claim 10, comprising about 5% by WCC of filler.

12. A cleansing composition according to claim 11, comprising about 3% to about 5% by WCC of filler.

13. A cleansing composition according to claim 6, wherein the first quantity of fragrance is X% less than the second quantity of fragrance expressed as a percentage by WCC and wherein the cleansing composition contains a quantity of filler expressed as a percentage by WCC that differs from X% by no more than about 3.

14. A cleansing composition according to claim 13, wherein the cleansing composition contains a quantity of filler expressed as a percentage by WCC that is substantially equivalent to X%.

15. A cleansing composition according to claim 6, wherein the cleansing composition has a first fragrance-to-surfactant ratio that is substantially equivalent to a second fragrance-to-surfactant ratio exhibited by the cleansing composition if the cleansing composition were to lack filler and contain the second quantity of fragrance.

16. A personal care cleansing composition having enhanced fragrance characteristics, the personal care cleansing composition comprising:
about 68% to about 83% by weight of the personal care cleansing composition (WCC) of surfactant;
about 10% to about 15% by WCC of water;
about 0.5% to about 2.0% by WCC of fragrance; and
about 1.0% to about 8.0% by WCC of talc.

17. A personal care cleansing composition according to claim 16, comprising:
about 1.0% to about 1.5% by WCC of fragrance; and
about 4.0% to about 6.0% by WCC of talc.

18. A personal care cleansing composition according to claim 17, comprising:
about 70% to about 80% by WCC of surfactant; and
about 11% to about 13% by WCC of water.

19. A personal care cleansing composition according to claim 18, wherein the personal care cleansing composition is substantially free of grease solvents.

20. A personal care cleansing composition according to claim 19, wherein the personal care cleansing composition is a toilet bar soap.
